(19) United States
(12) Patent Application Publication
(10) Pub. No.: US 2017/0281077 A1
(43) Pub. Date: Oct. 5, 2017

(54) SYSTEM WITH DISEASE DIAGNOSIS AND SKIN AGE MEASUREMENT FUNCTIONS AND HANDPIECE USED THEREWITH

(71) Applicant: Speclipse, Inc., Seoul (KR)

(72) Inventors: Sung Hyun PYUN, Seoul (KR); Wanki MIN, Gyeonggi-do (KR)

(73) Assignee: SPECLIPSE, INC.

(21) Appl. No.: 15/477,995

(22) Filed: Apr. 3, 2017

Related U.S. Application Data
(60) Provisional application No. 62/439,369, filed on Dec. 27, 2016.

(30) Foreign Application Priority Data
Apr. 4, 2016 (KR) 10-2016-0041316
Apr. 6, 2016 (KR) 10-2016-0041993
Sep. 8, 2016 (KR) 10-2016-0115669

(51) Int. Cl. A61B 5/00; A61N 5/06; A61B 18/20

(52) U.S. Cl. CPC A61B 5/4836; A61B 5/0075; A61B 5/0082; A61B 5/441; A61B 5/0013; A61B 18/203; A61N 5/0616; A61B 2017/00061

(57) ABSTRACT

An aesthetic or medical system is provided. The aesthetic or medical system includes: a laser irradiation device configured to generate a laser and project the laser onto body tissue; a probe configured to collect light which is generated when the laser is projected onto the body tissue; and an analysis unit configured to diagnose a disease by analyzing a spectrum of the light collected by the probe, and the probe is detachably connected to the laser irradiation device.

### CROSS-REFERENCE TO RELATED PATENT APPLICATION

This application claims benefits of and priorities to Korean Patent Applications No. 2016-0041316 filed on Apr. 4, 2016, No. 2016-0041993 filed on Apr. 6, 2016, and No. 2016-0115669 filed on Sep. 8, 2016 with the Korean Intellectual Property Office and U.S. Provisional Application No. 62/439,369 filed on Dec. 27, 2016 with the USPTO, the disclosures of which are incorporated herein by reference in its entirety.

### Field

Apparatuses and methods consistent with the exemplary embodiments relate to a system with disease diagnosis and skin age measurement functions and a handpiece used therewith.

### Background

Related-art laser irradiation devices such as a skin toning device, a skin peeling device, or a laser surgery device perform operations for medical or beauty care using lasers. From among the laser irradiation devices for medical or beauty care as described above, there are devices using collimated laser. The collimated laser has low energy density per unit area and thus devices using the collimated laser may not be used for other purposes than medical or beauty purposes.

Devices for measuring a skin age for the purpose of beauty or health are disclosed. For example, Korean Patent Publication No. 10-2015-0061914 (Jun. 5, 2015, titled “Method and Apparatus For Estimating Skin Age”) discloses the feature of measuring a skin.

### SUMMARY

One or more aspects of the exemplary embodiments provide an aesthetic or medical laser system which can diagnose a disease.

One or more aspects of the exemplary embodiments also provide an aesthetic or medical system which can diagnose a disease and measure a skin age.

One or more aspects of the exemplary embodiments also provide a handpiece which is combined with a laser irradiation device for use.

One or more aspects of the exemplary embodiments also provide a film which is used in an aesthetic or medical system which can diagnose a disease and measure a skin age, for increasing an emission spectrum.

According to an aspect of an exemplary embodiment, there is provided an system including: a laser irradiation device configured to generate a laser and project the laser onto body tissue; a probe configured to collect light which is generated when the laser is projected onto the body tissue; and an analysis unit configured to diagnose a disease by analyzing a spectrum of the light collected by the probe.

The laser projected onto the body tissue may be a collimated laser, and the probe may be detachably connected to the laser irradiation device.

According to an aspect of another exemplary embodiment, there is provided an handpiece detachably connected to a laser irradiation device, the handpiece including: a body part which is formed in a cylindrical shape to have a path formed therein to allow light to move therein, and which comprises an entrance part for receiving light generated when light is projected by the laser irradiation device onto body tissue, and a connection part which is attachable to or detachable from the laser irradiation device; and an optical unit which is configured to provide the generated light entering through the entrance part to a spectrometer.

According to one or more exemplary embodiments, a disease can be diagnosed using a laser irradiation device.

According to one or more exemplary embodiments, a disease can be diagnosed and a skin age can be measured using a laser irradiation device.

According to one or more exemplary embodiments, disease diagnosis and skin age measurement can be performed in addition to a medical or aesthetic process by simply connecting a handpiece according to an exemplary embodiment without changing the configuration of a related-art laser irradiation device.

According to one or more exemplary embodiments, even when the sensitivity of a signal of generated light is weak, a disease can be diagnosed and a skin age can be measured using an SELIBS film.

Additional aspects and advantages of the exemplary embodiments will be set forth in the detailed description, will be obvious from the detailed description, or may be learned by practicing the exemplary embodiments.

### BRIEF DESCRIPTION OF THE DRAWINGS

The above and other features and advantages will become more apparent by describing in detail exemplary embodiments with reference to the attached drawings in which:

FIG. 1 is a view to illustrate an aesthetic or medical system according to an exemplary embodiment of the present disclosure;

FIG. 2 is a view to illustrate an analysis unit according to an exemplary embodiment of the present disclosure;

FIG. 3 is a view to illustrate an analysis unit according to another exemplary embodiment of the present disclosure;

FIG. 4 is a view to illustrate an aesthetic or medical system according to another exemplary embodiment of the present disclosure;

FIG. 5 is a view to illustrate an aesthetic or medical system according to another exemplary embodiment of the present disclosure;

FIGS. 6A and 6B are views to illustrate a guide part according to another exemplary embodiment of the present disclosure;

FIG. 7 is a view to illustrate an aesthetic or medical system according to another exemplary embodiment of the present disclosure;

FIG. 8 is a view to illustrate an aesthetic or medical system according to another exemplary embodiment of the present disclosure;

FIGS. 9A and 9B are views to illustrate a detachable handpiece according to another exemplary embodiment of the present disclosure;

FIG. 10 is a view to illustrate a detachable handpiece according to another exemplary embodiment of the present disclosure; and

FIG. 11 is a view to illustrate a film which is used in an aesthetic or medical system, for increasing an emission spectrum according to an exemplary embodiment of the present disclosure.

### DETAILED DESCRIPTION

Exemplary embodiments will now be described more fully with reference to the accompanying drawings to clarify aspects, other aspects, features and advantages of the inventive concept. The exemplary embodiments may, however, be embodied in many different forms and should not be construed as limited to the exemplary embodiments set forth herein. Rather, the exemplary embodiments are provided so that this disclosure will be thorough and complete, and will fully convey the scope of the application to those of ordinary skill in the art.

It will be understood that when an element is referred to as being “on” another element, the element can be directly on another element or intervening elements. The terms “unit” and “module” and the terms having suffix “-er” or “-or” used in the following description refer to a unit for processing at least one function or operation, and may be implemented by hardware, software, or a combination of hardware and software.

Expressions such as “transmitting,” “communicating,” “receiving,” “providing,” or “forwarding” signals, data, or information, used in the following descriptions, or other expressions similar to the aforementioned expressions may refer to directly forwarding signals, data, or information from one element (“element a”) to another element (“element b”), and also refer to forwarding to element b via at least one other element (“element c”).

In the following description, elements “operatively related to each other” should be interpreted as being connected with each other in a wired and/or wireless manner so as to transmit and/or receive data. Although there is no explicit expression “an element (“element a”) and another element (“element b”) are operatively related to each other” in the description, if element a receives signals, data, or information outputted from element b and performs its operation (“element a”), or element b receives signals, data, or information outputted from element a and performs its operation (“element b”), it should be understood that element a and element b are “operatively related to each other.”

In the following description, a network may be configured by WiFi, Internet, a Local Area Network (LAN), a wireless LAN, a Wide Area Network (WAN), a Personal Area Network (PAN), 3G, 4G, LTE, a voice network, or a combination of two or more of the aforementioned networks.

The terms used herein are for the purpose of describing particular exemplary embodiments only and are not intended to limit the present disclosure. As used herein, the singular forms are intended to include the plural forms as well, unless the context clearly indicates otherwise. It will be further understood that the terms “comprises” and/or “comprising,” when used in this specification, do not preclude the presence or addition of one or more other components.

Definition of Terms

In the following description, the expression “a center axis of an oval cavity” refers to a virtual straight line passing through a first focal point (F1) and a second focal point (F2), and the expression “a center axis of a handpiece” refers to a virtual straight line passing through incident light which enters perpendicularly to a cut surface of the handpiece, and the second focal point (F2).

In addition, in the following description, the expression “the second focal point (F2) is substantially located on the cut surface” means that the second focal point (F2) is exactly located on the cut surface or the second focal point (F2) is located in the proximity of the cut surface. Herein, “the proximity of the cut surface” includes a location within a predetermined distance from the upper or lower portion of the cut surface. In addition, there is not a substantially big difference between a light receiving effect when the second focal point (F2) is located within the predetermined distance and a light receiving effect when the second focal point (F2) is exactly located on the cut surface. Thus, the predetermined distance may be defined as any distance in which the above condition is satisfied.

When a Surface Enhanced Laser Induced Breakdown Spectroscopy (SELIBS) film is disposed between the cut surface and body tissue, the expression “the second focal point (F2) is substantially located on the cut surface” may mean that the second focal point (F2) is located on the cut surface, the surface of the body tissue (i.e., a portion of the body tissue in contact with the SELIBS film), or the SELIBS film.

In addition, in the following description, the term “laser” means a pulsed laser, a continuous wave laser, a collimated laser, or a focused laser. In addition, the frequency band of the “laser” may have a certain frequency band, for example, an ultra violet (UV) band, a visible light band, or an infrared (IR) band.

In addition, the term “generated light” used in the following description encompasses all types of light which are generated when a laser is projected onto body tissue. Accordingly, the “generated light” may mean absorbed light, reflected light, scattered light, electronic emission light, and/or fluorescent light from the tissue.

In addition, in the following description, “an aesthetic or medical laser irradiation device” refers to a device which projects lasers and performs an operation for beauty or medical care. “A pulsed laser-based aesthetic or medical laser irradiation device” refers to a device which projects pulsed lasers and performs an operation for beauty or medical care.

In addition, in the following description, “a low level pulsed laser-based aesthetic or medical laser irradiation device” refers to a device which projects pulsed lasers and performs an operation for beauty or medical care, and which projects low level pulsed lasers such that it is difficult to use for other purposes except for medical or beauty purposes since energy density of the pulsed lasers per unit area is low.

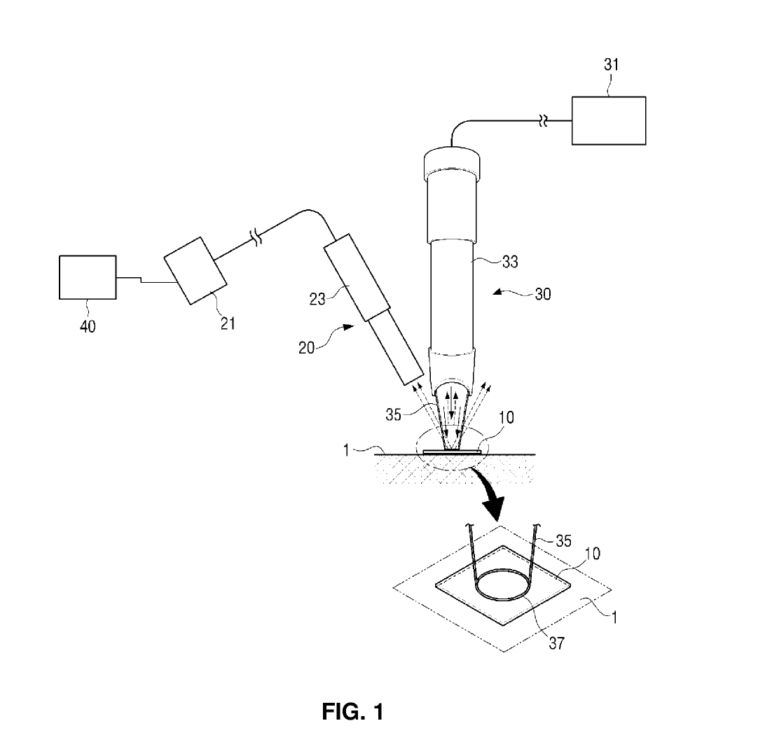

FIG. 1 is a view to illustrate an aesthetic or medical system according to an exemplary embodiment of the present disclosure.

Referring to FIG. 1, the system according to an exemplary embodiment of the present disclosure may be an aesthetic or medical system which can diagnose a disease (including cancer, and other different types of disease).

Referring to FIG. 1, the system according to an exemplary embodiment of the present disclosure may perform an operation for beauty care by projecting lasers on body tissue, and may determine whether there is a disease in body tissue by collecting light generated from the body tissue and analyzing a spectrum of the collected light.

Referring to FIG. 1, the aesthetic or medical system according to an exemplary embodiment of the present disclosure may include a Surface Enhanced Laser Induced Breakdown Spectroscopy (SELIBS) film 10, an analysis device 20, and a laser irradiation device 30. A display 40 is additionally illustrated for the purpose of explanation of FIG. 1.

In this embodiment, the laser irradiation device 30 is a device which performs an operation for medical or beauty care with respect to body tissue using lasers, and for example, may be a skin toning device, a skin peeling device, or a laser surgery device.

In this embodiment, the laser irradiation device 30 projects a laser onto body tissue 1 for the medical or aesthetic purpose. In one embodiment, the laser may be a collimated laser. The collimated laser may be referred to as parallel light or collimated light in the technical field to which the present disclosure belongs.

In this embodiment, the laser irradiation device 30 may be “an aesthetic or medical laser irradiation device”, “a low-level pulsed laser-based aesthetic or medical laser irradiation device”, or “a pulsed laser-based aesthetic or medical laser irradiation device”.

In this embodiment, the laser irradiation device 30 may include a laser handpiece 33 and a laser body 31 for generating a laser. The laser body 31 includes a laser generation source (not shown) for generating a laser, and the laser generated by the laser generation source (not shown) is provided to the handpiece 33. Although not shown, an optical device (for example, a collimator) for generating a collimated laser may be included in the laser body 31 or the handpiece 33.

In this embodiment, the laser irradiation device 30 may further include a guide part, and the guide part may help in projecting a laser emitted from the handpiece 33 onto a portion desired by the user. Herein, the guide part may be connected with the distal end of the handpiece 33.

In this embodiment, the guide part includes a ring 37 and a support part 35.

The analysis device 20 may diagnose a disease existing in the body tissue 1 by collecting light generated from the body tissue 1 (hereinafter, referred to as “generated light”) when the laser is projected onto the body tissue 1, and analyzing the spectrum of the collected light (hereinafter, referred to as “emission spectrum”).

The analysis device 20 diagnoses a disease by analyzing the spectrum of light. For example, U.S. Pat. No. 7,092,087 (Aug. 15, 2006) (hereinafter, ‘087’ patent) discloses a technical concept for diagnosing a disease by analyzing the spectrum of light. The feature disclosed in ‘087’ patent may be incorporated as a part of the description of the present application if the feature does not conflict with the concept of the present disclosure. The feature disclosed in this patent uses a principle that a specific element emits light of a specific wavelength when a laser is projected onto a specimen.

Additionally, the analysis device 20 may measure a skin age (e.g., the level of collagen or elastin concentration) by analyzing the spectrum of the collected light.

That is, when the body tissue 1 is a skin, the analysis device 20 may determine whether there is a disease and measure a skin age by analyzing the spectrum of the collected light.

The display 40 may display a result of the diagnosing by the analysis device 20. The display 40 may be a device provided with a monitor, for example, a mobile device such as a personal computer (PC), a smart phone, or a Personal Digital Assistant (PDA).

The analysis device 20 may include an analysis unit 21 and a probe 23.

In this embodiment, the probe 23 is configured to collect the light generated from the body tissue 1, and the analysis unit 21 may determine whether there is a disease or not and measure a skin age by analyzing the light collected by the probe 23. For example, the analysis unit 21 may determine whether there is a disease by analyzing the spectrum of the light collected by the probe 23 and comparing the analyzed spectrum and a database (DB) for disease diagnosis (hereinafter, referred to as “a reference spectrum data DB for disease diagnosis”) (e.g., a DB in which a specific disease matches the emission spectrum).

When the body tissue 1 is a skin, the analysis unit 21 may additionally measure a skin age. That is, the analysis unit 21 may measure the skin age in addition to determining whether there is a disease by analyzing the light collected by the probe 23. When measuring the skin age, the analysis unit 21 may measure the skin age by comparing the analyzed spectrum and a DB for measuring a skin age (hereinafter, referred to as “a reference spectrum data DB for skin age measurement”) (e.g., a DB in which the skin age matches the emission spectrum).

The light collected by the probe 23 when the skin age is measured may include plasma light, reflected light, scattered light, or fluorescent light, and the spectrum of the generated light may be changed due to a change in a chemical component such as collagen in the skin. The analysis unit 21 may measure the skin age by analyzing this spectrum.

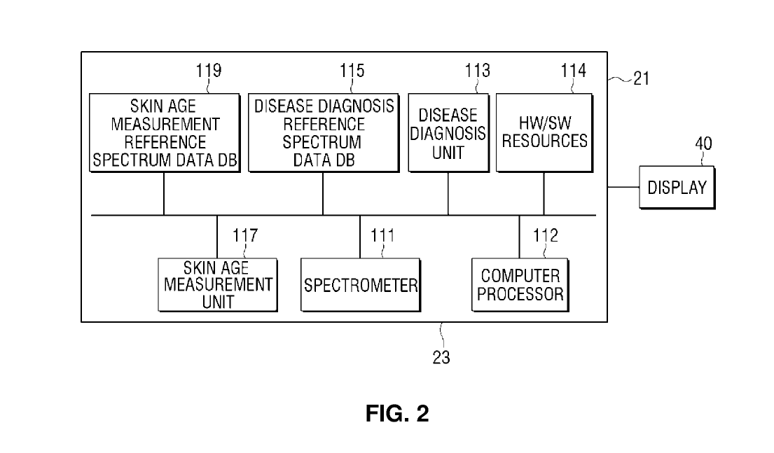

Hereafter, the operation of the analysis unit 21 will be described in detail with reference to FIG. 2.

FIG. 2 is a view to illustrate the analysis unit according to an exemplary embodiment of the present disclosure. Referring to FIG. 2, the analysis unit 21 according to an exemplary embodiment of the present disclosure may include a spectrometer 111, a computer processor 112, a disease diagnosis unit 113, HW/SW resources 114, a disease diagnosis reference spectrum data DB storage 115, a skin age measurement unit 117, and a skin age measurement reference spectrum data DB storage 119.

The HW/SW resources 114 refer to hardware and software which are required to perform the operation of the analysis unit 21.

In the following description, the term “HW/SW resources” is used for the purpose of explanation, and may be used to refer to all of hardware and software which are not directly related to achievement of the objects of the present disclosure.

For example, the HW/SW resources 114 may include a memory (not shown), a storage (not shown) for storing and reading data, image processing software (not shown) and hardware (not shown) for processing an image for displaying on the display 40, voice processing software (not shown) and hardware (not shown), transmitting and/or receiving units (not shown) for transmitting and/or receiving data from or to the outside, and programs for operating elements.

The computer processor 112 controls the elements of the analysis unit 21, for example, the spectrometer 111, the disease diagnosis unit 113, the HW/SW resources 114, the disease diagnosis reference spectrum data DB storage 115, the skin age measurement unit 117, and the skin age measurement reference spectrum data DB storage 119, to perform their respective operations.

The spectrometer 111 may receive the light collected by the probe 23 and measure the spectrum of the generated light.

The disease diagnosis unit 113 may determine whether there is a disease in the body tissue based on the spectrum measured by the spectrometer 111 with reference to the disease diagnosis reference spectrum data DB. For example, the disease diagnosis unit 113 may determine whether there is a disease by comparing the spectrum of the generated light measured by the spectrometer 111 and the disease diagnosis reference spectrum data DB.

The skin age measurement unit 117 may measure a skin age based on the spectrum measured by the spectrometer 111 with reference to the skin age measurement reference spectrum data DB. For example, the skin age measurement unit 117 may measure the skin age by comparing the spectrum of the generated light measured by the spectrometer 111 and the skin age measurement reference spectrum data DB. The skin age measurement unit 117 may identify a component for measuring the skin age through the spectrum of the generated light measured by the spectrometer 111. The component for measuring the skin age may be collagen or elastin, for example.

According to an exemplary embodiment, the skin age measurement reference spectrum data DB 119 may be a DB in which an amount of collagen or elastin matches a skin age. In this embodiment, the skin age measurement unit 117 may identify an amount of collagen or elastin through the spectrum of the generated light measured by the spectrometer 111 and may measure the skin age by referring to the skin age measurement reference spectrum data DB.

The result of the diagnosing by the disease diagnosis unit 113 and the result of the measuring by the skin age measurement unit 117 may be displayed on the display 40.

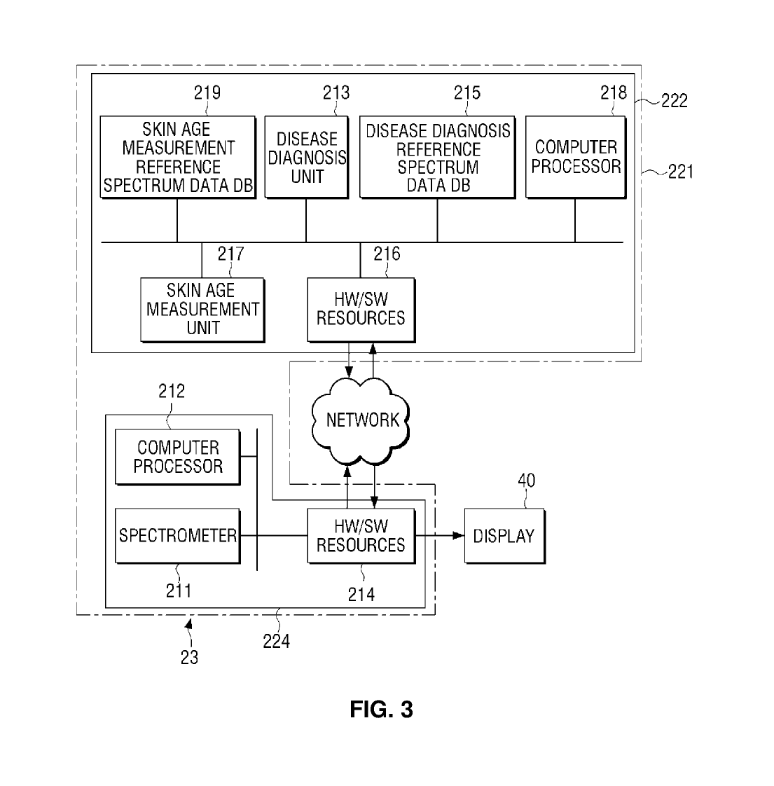

FIG. 3 is a view to illustrate an analysis unit according to another exemplary embodiment of the present disclosure.

Referring to FIG. 3, the analysis unit 221 according to another exemplary embodiment of the present disclosure may include a diagnosis server 222 and a diagnosis terminal 224. Herein, the diagnosis server 222 and the diagnosis terminal 224 may be operatively connected with each other through a network.

The diagnosis server 222 may include a disease diagnosis unit 213, a disease diagnosis reference spectrum data DB storage 215, a skin age measurement unit 217, a skin age measurement reference spectrum data DB storage 219, HW/SW resources 216, and a computer processor 218.

The diagnosis terminal 224 may include a spectrometer 211, a computer processor 212, and HW/SW resources 214. The diagnosis terminal 224 may transmit the spectrum of the light collected by the probe 23 to the diagnosis server 222, and receive the results of diagnosing and measuring from the diagnosis server 222. The result of the diagnosing and the measuring may be displayed by the display 40.

The HW/SW resources 214 refer to hardware and software required to perform the operation of the diagnosis terminal 224. For example, the HW/SW resources 214 may include a memory (not shown), a storage (not shown) for storing and reading data, image processing software (not shown) and hardware (not shown) for processing an image for displaying on the display 40, voice processing software (not shown) and hardware (not shown), transmitting and/or receiving units (not shown) for transmitting and/or receiving data from or to the outside, and programs for operating elements.

The HW/SW resources 216 refer to hardware and software required to perform the operation of the diagnosis server 222. For example, the HW/SW resources 216 may include a memory (not shown), a storage (not shown) for storing and reading data, transmitting and/or receiving units (not shown) for transmitting and/or receiving data from or to the outside, and programs for operating elements.

The computer processor 212 controls the elements of the diagnosis terminal 224, for example, the spectrometer 211 and the HW/SW resources 214, to perform their respective operations, and the computer processor 218 controls the elements of the diagnosis server 222, for example, the disease diagnosis unit 213, the disease diagnosis reference spectrum data DB storage 215, the skin age measurement unit 217, and the skin age measurement reference spectrum data DB storage 219, and the HW/SW resources 216, to perform their respective operations.

Referring to FIG. 3, the spectrometer 211 measures the spectrum of the light collected by the probe 224, and transmits the spectrum measured by the spectrometer 211 to the diagnosis server 222 via a network.

The diagnosis server 222 analyzes the spectrum received from the diagnosis terminal 224 and diagnoses a disease, and transmits the result of the measuring a skin age to the diagnosis terminal 224 via the network. The result received by the diagnosis terminal 224 is displayed by the display 40.

The operation of the diagnosis server 222 will be described in detail.

The diagnosis server 222 may receive the spectrum measured by the spectrometer 211, and the disease diagnosis unit 213 may determine whether there is a disease by comparing the received spectrum and the disease diagnosis reference spectrum data DB. In addition, the skin age measurement unit 217 may measure the skin age by comparing the received spectrum and the skin age measurement reference spectrum data DB.

The diagnosis server 222 transmits the result of the diagnosing by the disease diagnosis unit 213 and the result of the measuring by the skin age measurement unit 217 to the diagnosis terminal 224 via the network. Thereafter, the results transmitted to the diagnosis terminal 224 are displayed by the display 40.

In the above description, the analysis unit according to exemplary embodiments of the present disclosure has been described with reference to FIGS. 2 and 3. In the embodiments, the analysis unit includes the skin age measurement unit 117 or 217 and the skin age measurement reference spectrum data DB. However, the skin age measurement unit 117 or 217 and the skin age measurement reference spectrum data DB may not be included. That is, the analysis unit according to the exemplary embodiments of the present disclosure may be configured to diagnose a disease without measuring a skin age.

Referring back to FIG. 1, other elements will be described.

The light collected by the probe 23 is light which is generated from the body tissue 1 when the laser is projected onto the body tissue 1, and which passes through the SELIBS film 10. The diffused light generated from the body tissue 1 increases its intensity while passing through the SELIBS film 10.

In this embodiment, the SELIBS film 10 has one surface or both surfaces configured to increase the intensity of light, and an adhesive may be formed on one surface of the film 10 to temporarily attach the film 10 to the body tissue 1. The forming the adhesive on the film 10 is optional. Accordingly, a person skilled in the art may use the adhesive or may not use according to his/her selection.

The output of the laser projected onto the body tissue 1 by the laser irradiation device 30 is low. This is because strong output may cause damage to the body tissue 1. Accordingly, the laser irradiation device 3 which is used commercially projects the laser having low output not to cause damage to the body tissue onto the body tissue. When the laser is projected onto the body tissue 1, the intensity of light generated from the body tissue 1 is too weak to analyze the spectrum and thus the light may not be used in the analysis device 20.

However, in the embodiments, the SELIBS film is used such that the analysis device 20 can be used although the laser irradiation device 3 using the low level laser is used. Therefore, the aesthetic or medical system according to an exemplary embodiment of the present disclosure may perform the operation for medical or beauty care with respect to the body tissue 1, and simultaneously, may diagnose a disease and measure a skin age.

As will be described below, the SELIBS film may be used in other embodiments which will be described below.

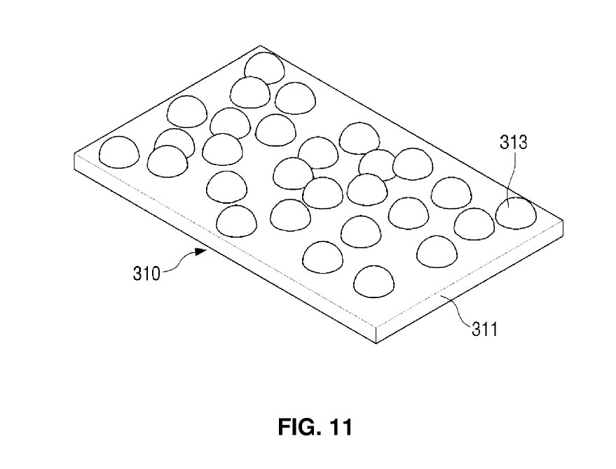

The SELIBS film 10 is configured to have a function of increasing a signal intensity of light generated from the body tissue 1. FIG. 11 illustrates an example of a structure of the SELIBS film 10.

FIG. 11 is a view to illustrate a film for increasing the intensity of generated light used in the aesthetic or medical system according to an exemplary embodiment of the present disclosure.

Referring to FIG. 11, an SELIBS film 310 may be configured to include a substrate 311 and a microscopic structure 313. Herein, the substrate 311 may be formed of polymer, and the microscopic structure 313 is for increasing a signal intensity light generated from body tissue when a laser is projected onto the body tissue, and for example, may be metal particles having a nano or micro size (a few nanometers to a few hundred micrometers).

In another example, the film 310 may be formed of metal having surface roughness of a nano or micro size (a few nanometers to a few hundred micrometers).

In the Raman spectroscopy, a technology for increasing the intensity of light scattered when a laser is projected (Surface Enhanced Raman Scattering (SERS)) is used. In the SERS, the scattering light generated when the laser is projected increases and the Raman analysis is performed. For example, the paper “Towards low-cost flexible substrates for nanoplasmonic sensing” (Phys. Chem. Chem. Phys., 2013, 15, 5288-5300) discloses an element for increasing the intensity of a Raman signal.

The SELIBS film used in the exemplary embodiments of the present disclosure may be produced by utilizing the related-art element for increasing the intensity of the Raman signal or by utilizing an appropriate element for increasing the intensity of generated light through experiments.

The SELIBS film used in the exemplary embodiments of the present disclosure may be formed in various shapes.

For example, an appropriate substrate (hereinafter, referred to as an “SELIBS substrate”) which may be used as the SELIBS film is prepared, and the surface of the SELIBS substrate is processed using a physical or chemical method to have surface roughness of a nano or micro size (a few nanometers to a few hundred micrometers).

In another example, a method for temporarily or fixedly forming a layer (hereinafter, referred to as an increasing layer) for increasing the intensity of generated light on the surface of the SELIBS substrate may be used. Herein, the signal intensity increasing layer may be formed of material including a microscopic structure, for example.

For example, according to the method for temporarily forming, the increasing layer is formed by spraying material for increasing the intensity of generated light onto the surface of the SELIBS substrate or coating the surface with the material. The SELIBS film formed in this method may be required to be cleaned after being used in the medical or aesthetic system according to an exemplary embodiment of the present disclosure, and material having a microscopic structure may be sprayed or coated on the surface of the cleaned SELIBS substrate again, such that the cleaned SELIBS substrate can be reused.

For example, according to the method for fixedly forming, the increasing layer is formed by spraying material having a microscopic structure onto the surface of the SELIBS substrate or coating the surface with the material, and then curing the SELIBS substrate.

The medical or aesthetic system according to an exemplary embodiment of the present disclosure may not use the SELIBS film and may apply a method for directly spraying or coating material having a microscopic structure onto or over body tissue. That is, the system may directly spray or coat material (material for increasing the intensity of generated light) having a microscopic structure onto or over body tissue, and may project a laser and collect generated light in this state. The method for forming the SELIBS film and the method for directly spraying or coating the material having the microscopic structure onto or over the body tissue without using the SELIBS film can be applied to embodiments which will be described below with reference to the other drawings.

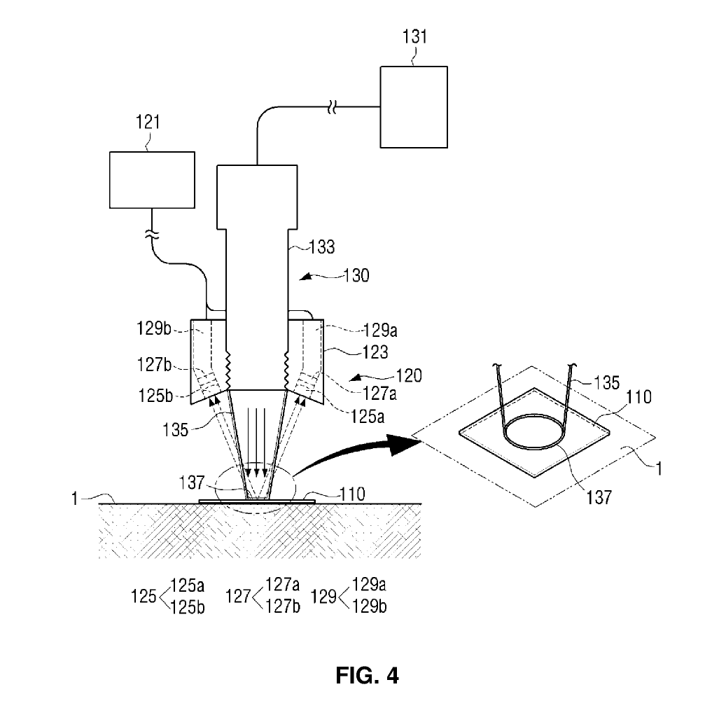

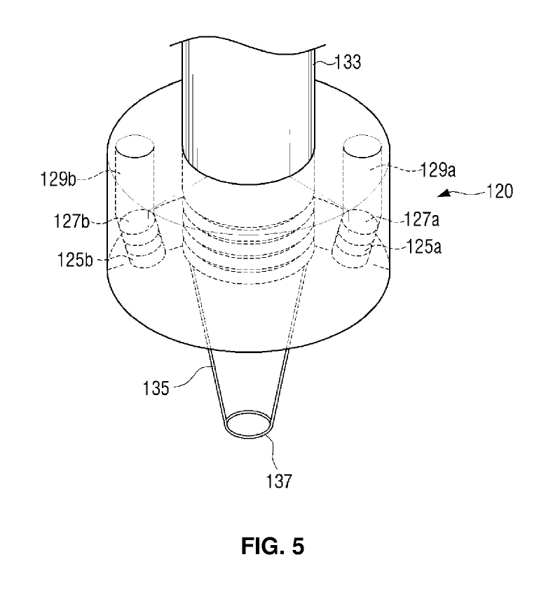

FIGS. 4 and 5 are views to illustrate an aesthetic or medical system according to another exemplary embodiment of the present disclosure.

Referring to FIGS. 4 and 5, the aesthetic or medical system according to another exemplary embodiment of the present disclosure may include a laser irradiation device 130, an analysis device 120, and an SELIBS film 110.

The analysis device 120 may include an analysis unit 121 and a probe 123. Herein, the analysis unit 121 may be the analysis unit which has been described above with reference to FIGS. 1 to 3.

Comparing the embodiment of FIG. 1 and the embodiment of FIG. 4, there is a difference in that the probe 123 and the laser irradiation device in the embodiment of FIG. 4 are detachably connected with each other.

Referring to FIGS. 4 and 5, the probe 123 is connected to the laser irradiation device 130. For example, the probe 123 may be connected to the laser irradiation device 130 by means of screw connection to surround the laser irradiation device 130 as shown in the drawings. The screw connection is merely an example, and the probe 123 and the laser irradiation device 130 may be connected with each other by means of other connection means.

In this embodiment, the laser irradiation device 130 may be “an aesthetic or medical laser irradiation device”, “a low level pulsed laser-based aesthetic or medical laser irradiation device”, or “a pulsed laser-based aesthetic or medical laser irradiation device.”

In this embodiment, the laser irradiation device 130 may include a handpiece 133 and a laser body 131 for generating lasers.

In this embodiment, the probe 123 may be formed in a substantially cylindrical shape having a cavity formed therein, and the handpiece 133 of the laser irradiation device 130 is inserted into the cavity and connected with the probe 123. Due to this connection method, the probe 123 is configured to easily receive light generated by the laser projected onto the body tissue 1 through the handpiece 133.

A structure for screwing and a guide part may be formed at the distal end of the handpiece 133. The guide part is to provide convenience to the user by allowing the laser emitted from the handpiece 133 to be projected onto an appropriate location.

In this embodiment, the guide part includes a ring 137 and a support part 135. The support part 135 may maintain the ring 137 at a distance from the main body of the handpiece 133. The support part 135 connects the ring 137 and the distal end of the handpiece 133, but makes the ring 137 and the distal end of the handpiece 133 spaced from each other. The support 135 may be formed of material like a wire.

The laser irradiation device 130 has the same or similar functions as or to those of the laser irradiation device 30 described with reference to FIG. 1, except for that the laser irradiation device 130 is connected with the probe 123.

The analysis device 120 may collect light generated from the body tissue 1 when the laser is projected onto the body tissue 1, and may diagnose a disease existing in the body tissue 1 by analyzing the spectrum of the collected light and may measure a skin age when the body tissue 1 is a skin.

The analysis device 120 may diagnose a disease by analyzing the spectrum of light, and for example, may diagnose a disease by using Laser Induced Breakdown Spectroscopy (LIBS). Herein, the LIBS is merely an example and other spectroscopy methods can be applied.

The analysis device 120 may measure a skin age by referring to a DB (not shown) for measuring a skin age (a DB in which the skin age matches the emission spectrum).

In this embodiment, the probe 123 may collect the light generated from the body tissue 1, and the analysis unit 121 may determine whether there is a disease by analyzing the light collected by the probe 123 and may measure a skin age when the body tissue 1 is a skin. Herein, the light actually collected by the probe 123 may be light which is generated from the body tissue 1 when the laser is projected onto the body tissue, and which passes through the SELIBS film 110. The light generated from the body tissue 1 increases the intensity of a signal while passing through the SELIBS film 110. A structure for increasing the intensity of generated light may be formed on at least one surface of the SELIBS film 110, and an adhesive may be formed on one surface of the film 110 to temporarily attach the film 110 to the body tissue.

Referring to FIGS. 4 and 5, in this embodiment, the probe 123 may include a lens 125 (125a, 125b) for collecting generated light, a filter 127 (127a, 127b) for passing therethrough only a predetermined frequency band of the light collected by the lens 125 (125a, 125b), and an optical fiber 129 (129a, 129b) for providing a path through which the collected light moves.

In this embodiment, the lens 125 may include a first lens 125a and a second lens 125b, the filter 127 may include a first filter 127a and a second filter 127b, and the optical fiber 129 may include a first optical fiber 129a and a second optical fiber 129b. Herein, the first lens 125a and the second lens 125b may have the same function, the first filter 127a and the second filter 127b may have the same function, and the first optical fiber 129a and the second optical fiber 129b may have the same function. Although two lenses, two filters, and two optical fibers are included in this embodiment, this is merely an example. The probe 123 may include a single lens, a single filter, and a single optical fiber (that is, the first lens, the first filter, and the first optical fiber) or may include three or more lenses, three or more filters, and three or more optical fibers.

In the system described above with reference to FIGS. 4 and 5, the probe 123 is detachably connected to the laser irradiation device 130. However, this is merely an example. The probe 123 and the laser irradiation device 130 may be fixedly connected with each other. In this case, the elements 125, 127, and 129 included in the probe 123 may be disposed inside the handpiece 133.

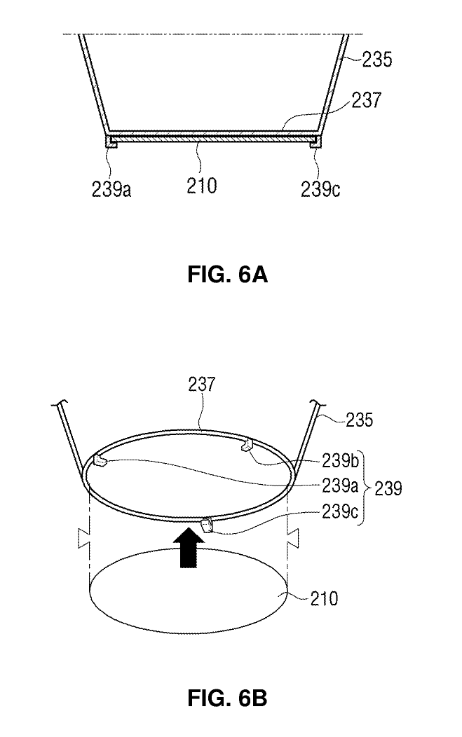

FIGS. 6A and 6B is views to illustrate a guide part according to another exemplary embodiment of the present disclosure.

FIG. 6A is a cross section view of the guide part according to another exemplary embodiment of the present disclosure, and FIG. 6B is a view showing an SELIBS film 210 which is mounted in the guide part

Referring to FIGS. 6A and 6B, the guide part according to an exemplary embodiment of the present disclosure includes a ring 237 and a support part 235. The guide part according to FIGS. 6A and 6B may be used after being combined to the handpiece 33 depicted, for example, in FIG. 1. That is, the guide part described with reference to FIGS. 6A and 6B may be substituted for the guide part used in the handpiece 33 of FIG. 1.

The guide part according to FIGS. 6A and 6B may be connected with the distal end of the handpiece 133 described with reference to FIGS. 4 and 5. That is, the guide part described with reference to FIGS. 6A and 6B may be substituted for the guide part used in the handpiece 133 of FIGS. 4 and 5.

The support part 235 may maintain the ring 237 at a distance from the main body of the handpiece. The support part 235 may connect the ring 237 and the distal end of the handpiece, but may make the ring 237 and the distal end of the handpiece spaced from each other by a predetermined distance.

In this embodiment, a film attachment/detachment support part 239 for detachably connecting the SELIBS film 210 may be formed at the distal end of the handpiece. For example, the film attachment/detachment support part 239 may be formed on the guide part which is formed at the distal end of the hand piece. Specifically, the film attachment/detachment support part 239 may be formed on the ring 237 of the guide part. Referring to views (a) and (b) of FIG. 6, the principle that the SELIBS film 210 is detachably connected with the ring 237 could be understood. Herein, the shape or number of the film attachment/detachment support parts 239 is merely an example and may be provided differently.

The guide part described above with reference to FIG. 6 may be used instead of the guide part illustrated in the embodiments of FIGS. 1, 4, and 9.

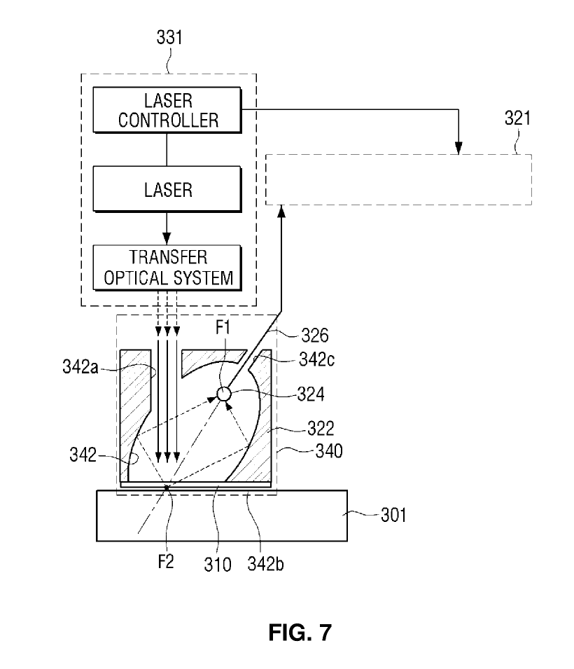

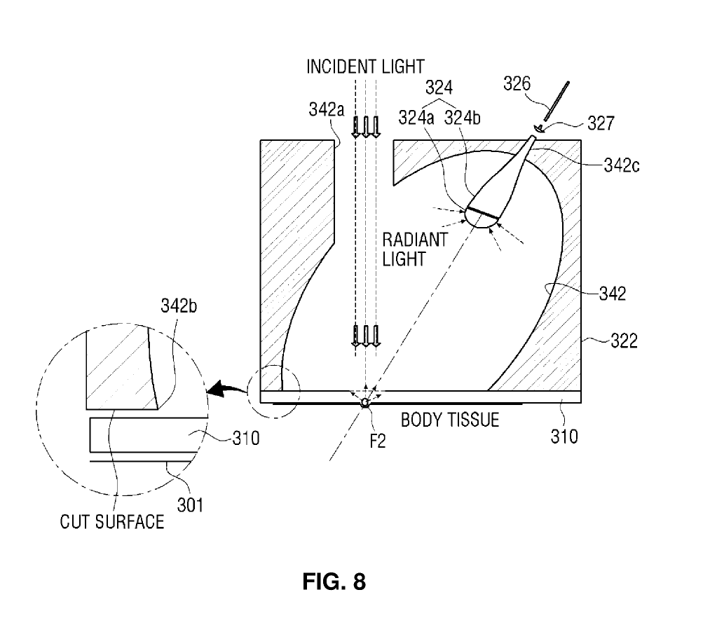

FIGS. 7 and 8 are views to illustrate a system according to another exemplary embodiment of the present disclosure.

Referring to FIGS. 7 and 8, the system according to another exemplary embodiment of the present disclosure may include a laser irradiation device, an analysis unit, and an SELIBS film 310.

As shown in an enlarged view of FIG. 8, a cut surface, a part given reference numeral 310, and a part given reference numeral 301 are spaced from one another. However, this is merely an example for the purpose of explanation. It would be easily understood by a person skilled in the art that, when the medical or aesthetic system is actually used, the cut surface, the part given reference numeral 310, and the part given reference numeral 301 may be in contact with one another.

In this embodiment, the laser irradiation device may be “an aesthetic or medical laser irradiation device”, “a low level pulsed laser-based aesthetic or medical laser irradiation device”, or “a pulsed laser-based aesthetic or medical laser irradiation device.”

In this embodiment, the laser irradiation device may include a handpiece 340 for diagnosing a disease and measuring a skin age (hereinafter, referred to as a handpiece 340), and a laser body 331, and the analysis unit may determine whether there is a disease or not by analyzing the spectrum of light generated from body tissue 301.

The laser body 331 may include a laser controller, a laser generation source for generating a laser, and a transfer optical system. The laser controller may control the laser generation source and may control the operation of a spectrometer included in the analysis unit.

According to the exemplary embodiment described with reference to FIGS. 7 and 8, the analysis unit may additionally measure a skin age by analyzing the spectrum of light generated from the body tissue 301.

The operation and configuration of the analysis unit according to the exemplary embodiment described with reference to FIGS. 7 and 8 are the same as those of the embodiments described with reference to FIGS. 1 to 3. That is, the analysis unit according to various exemplary embodiments described above with reference to FIGS. 1 to 3 may be used as the analysis unit according to the embodiment described with reference to FIGS. 7 and 8.

In this embodiment, the handpiece 340 may be used to project a laser for medical or beauty care onto the body tissue, and simultaneously, may collect generated light for diagnosing a disease and measuring a skin age. That is, the handpiece 340 may receive the laser from the laser body 331 and project the laser onto the body tissue, and simultaneously, may collect a light generated from the body tissue and forward the collected light to the analysis unit 321.

In this embodiment, the SELIBS film 310 may be located between the body tissue 301 and the handpiece 340. For example, the SELIBS film 310 may be fixed to or detachably connected to the distal end of the handpiece 340, or may be inserted between the handpiece 340 and the body tissue 301 without being connected with the handpiece 340.

In this embodiment, the laser body 331 outputs a pulsed laser. For example, the laser may be a CO2 laser, a Nd:YAG laser, an excimer laser, or an argon laser. The pulse width of the pulsed laser has pulse duration of a few fs (picoseconds) to ns (nanoseconds), and the output of the pulsed laser may be 1 GW/cm2 or higher. The laser controller may control the laser, and may generate a pulse signal when light is outputted and provide the pulse signal to the spectrometer.

In this embodiment, the transfer optical system converts the laser outputted from the laser generation source into a collimated laser or a focused laser. Alternatively, the transfer optical system may transmit the laser light outputted from the laser to the handpiece 340 as it is.

The handpiece 340 receives the laser via the transfer optical system and projects the laser onto the body tissue 301, and collects a light generated from the body tissue 301. The light collected by the handpiece 340 is provided to the analysis unit 321 via an optical fiber 326.

The analysis unit 321 is the same as in the embodiments described above with reference to FIGS. 1 to 3.

The handpiece 340 includes a body part 322 having an oval cavity 342 formed therein, and a light collection unit 324 disposed on a first focal point (F1) of the oval cavity 342 to collect the light reflected from the oval cavity 342.

The body part 322 is provided with a cut surface which is not perpendicular to the center axis (or major axis) of the oval cavity 342.

In this embodiment, a second focal point (F2) of the oval cavity 342 is substantially located on the cut surface.

For example, when the cut surface is located in contact with the body tissue 301, the second focal point (F2) of the oval cavity 342 is placed on the body tissue 301.

When the SELIBS film 310 is placed between the cut surface and the body tissue 301, the second focal point (F2) may be located on the cut surface, the SELIBS film 310, or the surface of the body tissue 301 (a portion in contact with the SELIBS film).

The handpiece 340 may project the laser onto the body tissue 301. The user may project the laser onto a desired location by gripping the handpiece 340 with user's hand. The body tissue 301 may be a human body skin, but is not limited to this.

The oval cavity 342 may have an inner surface or an oval surface coated with metal so as to reflect the generated light.

The oval cavity 342 is provided with the first focal point (F1) and the second focal point (F2) as described above. The eccentricity of the oval cavity 342 may be about 0.5 to 0.9, for example. The light generated at the first focal point in a radial pattern converges on the second focal point. The handpiece 340 may be formed in a cylindrical shape, for example, but is not limited to this. In this embodiment, the center axis of the handpiece 340 and the center axis of the oval cavity 342 are not parallel to each other and form a predetermined angle therebetween. Herein, the predetermined angle may be an acute angle less than 90°, for example.

When the point at which the light is generated is the second focal point (F2), the light of all paths reflected from the oval cavity is collected at the first focal point (F1). When the light collection unit 324 is disposed at the first focal point (F1), the generated light can be effectively collected. Accordingly, since a larger amount of light is transmitted to the spectrometer in comparison to the related-art methods, spectrum signal sensitivity can be enhanced.

The body part 322 may include a laser inlet 342a, a laser emitter 342b, and a generated light outlet 342c.

The laser inlet 342a is a portion through which the laser enters from the laser body 331, the laser emitter 342b is a portion through which the laser entering via the laser inlet 342a is emitted to the body tissue, and the generated light outlet 342c is a portion through which the light collected by the light collection unit 324 is outputted to the outside. Herein, the laser inlet 342a and the laser emitter 342b are wide and are shaped enough to allow the laser to pass therethrough, and are aligned with each other such that the laser passes in a straight line.

The cut surface provides a plane to be brought into contact with the body tissue 301, and may substantially pass through the second focal point. The laser emitter 342b may be formed on the cut surface.

The laser inlet 342a is located such that the light path of the laser entering through the laser inlet 342a and the center axis (or major axis) of the oval cavity 342 are not parallel to each other and cross at a predetermined angle. Herein, the predetermined angle may be an acute angle less than 90°. For example, when the body part 322 is formed in a cylindrical shape, the laser inlet 342a is formed on the top surface of the cylinder and the cut surface is formed on the bottom surface of the cylinder, and the generated light outlet 342c may be formed on the top surface of the cylinder. The generated light outlet 342c may be placed on an extension line of the center axis of the oval cavity.

According to an exemplary embodiment of the present disclosure, an angle between the laser perpendicularly entering the cut surface through the laser inlet 342a and the center axis of the oval cavity 342 may be 5° to 45°. The laser inlet 342a and the laser emitter 342b are aligned with each other such that the laser entering through the laser inlet 342a enters the cut surface perpendicularly.

The light in the radial pattern is collected at the first focal point (F1). An optical system is required to transmit the collected light to an optical fiber having a limited numeral aperture (NA) without loss.

For example, the light collection unit 324 may include a hemispherical lens 324a and a tapered optical fiber optic plate 324b. The spherical surface of the hemispherical lens 324a may be located on the center axis of the oval cavity 342. Herein, the spherical surface of the hemispherical lens 324a may be located in a direction toward the second focal point (F12). Preferably, the center of the spherical surface of the hemispherical lens 324a may be located on the center axis of the oval cavity 342.

The tapered optical fiber optic plate 324b may be disposed in contact with the plane of the hemispherical lens 324a.

The light collected at the first focal point is refracted through the hemispherical lens 324a and an incidence angle of light entering the tapered optical fiber optic plate 324b is reduced. The tapered optical fiber optic plate 324b may have several tens to several hundreds of optical fiber bundles compressed therein and may be tapered. Accordingly, the tapered optical fiber optic plate 324b receives light through one end having a large diameter, and transmits the light through the other end having a small diameter without causing additional light loss and distortion.

One end of the tapered optical fiber optic plate 324b may be disposed in contact with the plane of the hemispherical lens 324a, and the other end of the tapered optical fiber optic plate 324b may be disposed in the generated light outlet 342c.

The light transmitted through the other end of the tapered optical fiber optic plate 324b may be transmitted to the optical fiber 326 via a focusing lens 327. The focusing lens 327 is a plane-convex structure and has the plane disposed toward the optical fiber. Accordingly, the generated light proceeds along the optical fiber without loss.

Normally, the light generated as a result of the laser projection generates minute craters in the laser proceeding direction. Accordingly, the generated light has high optical density in the laser proceeding direction rather than a complete radial direction. For that reason, in the related-art method, the generated light is collected by the focusing lens via the same path as the entering path of the laser projected onto the body tissue. However, the generated light may be lost in an area which is not covered by the focusing lens, and there is a need for a method for collecting lost light as much as possible in order to enhance spectrum signal sensitivity.

According to the embodiments described above with reference to FIGS. 7 and 8, in the oval cavity having a reflection surface formed therein, the light collection unit may be disposed at the first focal point and the body tissue may be placed on the second focal point. In this case, when a laser is projected onto the body tissue of the second focal point, light is generated from the body tissue. In this case, the generated light is reflected and collected at the first focal point, and the light collection unit disposed at the first focal point effectively collects the light and thereby enhances signal sensitivity. In particular, this structure increases the light collection efficiency. In addition, the oval reflector is tilted with reference to the incident laser. Accordingly, the light path of the incident laser is separated from the light path of the generated light. Due to the separation of the light path, the light collection efficiency can be enhanced.

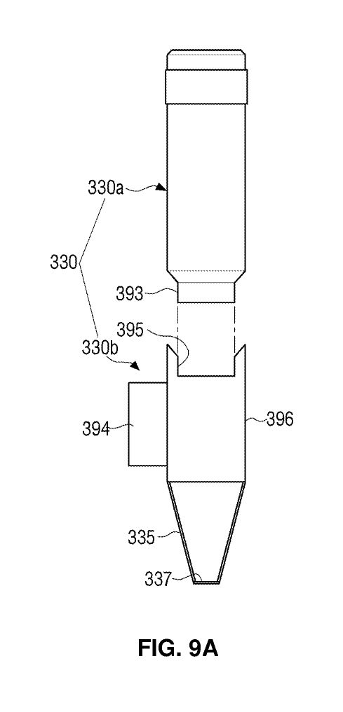

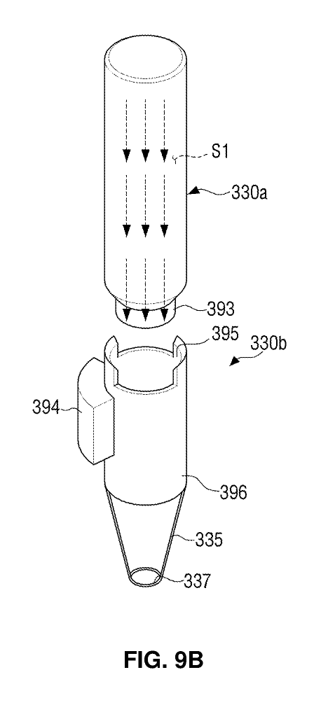

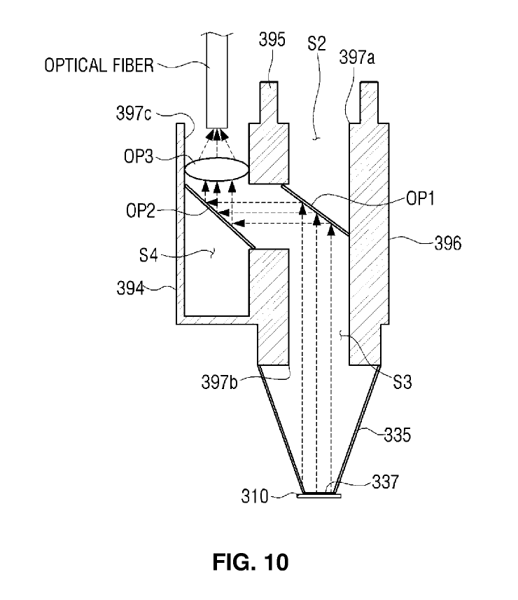

FIGS. 9A, 9B, and 10 are views to illustrate a detachable handpiece according to another exemplary embodiment of the present disclosure.

Referring to FIGS. 9A, 9B, and 10, the detachable handpiece 330b connected with a laser irradiation device according to an exemplary embodiment (hereinafter, referred to as “a detachable handpiece”) is detachably connected to a handpiece 330a provided in the laser irradiation device and is used. Herein, the laser irradiation device is a device for projecting a laser onto body tissue and is provided with the handpiece 330a.

In this embodiment, the laser irradiation device may be “an aesthetic or medical laser irradiation device”, “a low level pulsed laser-based aesthetic or medical laser irradiation device”, or “a pulsed laser-based aesthetic or medical laser irradiation device.”

According to an exemplary embodiment of the present disclosure, the detachable handpiece 330b is connected to be attachable to or detachable from the handpiece 330a provided in the laser irradiation device.

According to an exemplary embodiment of the present disclosure, the detachable handpiece 330b and the handpiece 330a provided in the laser irradiation device are connected with each other, thereby forming the handpiece 330 in the form of one piece. The handpiece 330 in the form of one piece may provide convenience to the user.

According to an exemplary embodiment, the handpiece 330 in the form of one piece which is formed by connecting the detachable handpiece 330b and the handpiece 330a provided in the laser irradiation device may be substituted for the handpiece 30 described with reference to FIG. 1.

As will be explained blow, an optical fiber of the handpiece 330 implemented in the form of one piece is connected to an analysis unit (for example, the analysis unit 21 described with reference to FIGS. 1 to 3), and light provided from a laser body (for example, the laser body 31 described with reference to FIG. 1) enters the handpiece 330 implemented in the form one piece.

According to an exemplary embodiment, the handpiece 330 in the form of one piece which is formed by connecting the detachable handpiece 330b and the handpiece 330a provided in the laser irradiation device may be substituted for the handpiece 133 described with reference to FIG. 4.

According to an exemplary embodiment, the handpiece 330b includes a body part 396 having spaces S2 and S3 formed therein, for allowing light to move therein. The light moving in the body part 396 may be a laser provided from the laser irradiation device or may be light generated from body tissue.

The body part 396 may include a connection part 395, a laser inlet 397a for receiving a laser from the laser irradiation device, a laser emitter 397b for emitting the laser entering through the laser inlet 397a to body tissue, and a generated light outlet 397c through which generated light is outputted to the outside.

Herein, the laser inlet 397a and the laser emitter 397b are wide and are shaped enough to allow the laser to pass therethrough, and are aligned with each other such that the laser passes therethrough. In addition, the generated light outlet 397c may be connected to an optical fiber to provide the generated light to the optical fiber.

The body part 396 may be formed in a cylindrical shape so as to allow the light to move therein, and the connection part 395 may be formed at one end of the cylinder and the laser emitter 397b may be formed at the other end of the cylinder.

The connection part 395 may be detachably connected with a connection part 393 of the handpiece 330a of the laser irradiation device. For example, a screw structure may be formed on the connection part 395 and the connection part 393 to connect them each other.

In this embodiment, the light generated from the body tissue enters the inner space S3 of the body part 396 through the laser emitter 397b. That is, the laser provided from the laser irradiation device is projected through the laser emitter 397b and the light generated from the body tissue enters the body part 396 through the laser emitter 397b.

An optical unit located in the body part 396 provides at least part of the generated light entering through the laser emitter 397b to the analysis unit (for example, the analysis unit 21 or the analysis unit 121).

In this embodiment, the optical unit may include an optical module OP1 for changing the direction of the generated light entering through the laser emitter 397b, an optical module OP2 for re-changing the direction of the generated light of which the direction has been changed by the optical module OP1, and an optical module OP3 for transmitting the generated light of which the direction has been re-changed by the optical module OP2 to the optical fiber connected with the analysis unit (for example, the analysis unit 21 or the analysis unit 121).

In this embodiment, the optical module OP1 may be an optical device which changes the direction of the generated light entering through the laser emitter 397b by 90°, and the optical module OP2 may be an optical device which changes the direction of the generated light of which the direction has been changed by the optical module OP1 by 90°. The optical module OP3 may be an optical device which provides the generated light of which the direction has been changed by the optical module OP2 to the optical fiber like a lens.

In this embodiment, the body part 396 may include a first part, a second part, and a third part, and these parts provide paths through which light moves. The first part is where the first optical module OP1 is located, the second part is where the optical modules OP2 and OP3 are located, and the third part is a guide part.

In this embodiment, the first part is formed in a cylindrical shape such that it can receive the laser provided from the laser irradiation device and emit the laser to the body tissue as it is. The connection part 395 and the laser inlet 397a may be formed at one end of the first part and the laser emitter 397b may be formed at the other end of the first part. In exemplary embodiments, the part given reference numeral 397 may be referred to as a “entrance part” or a “laser emitter” in the description. This is because the light generated from the body tissue may enter through the part given reference numeral 397 or the laser provided from the laser irradiation device may be emitted to the body tissue through the part given reference numeral 397.

In this embodiment, the entrance part and the laser emitter are located at the same location, but this is merely an example. It would be easily understood by a person skilled in the art that the entrance part and the laser emitter may be located at different locations.

In this embodiment, the laser provided from the laser irradiation device enters the first part where the laser inlet 397a is formed, and the entering laser is projected onto the body tissue.

In this embodiment, the first part is located at the center of the body part 396, and the second part is located to enclose the first part.

According to an exemplary embodiment, the handpiece 330b may further include a guide part. The guide part forms the third part as described above, and is connected with the entrance part 397 of the body part 396. The guide part includes a ring 337 and a support part 335. The guide part guides the user to project the laser outputted through the entrance part 397 onto a portion desired by the user. For example, the guide part may be configured to allow the laser outputted through the laser emitter 397b to pass through the center of the ring 337. Therefore, the user may place the center of the ring 337 on a portion desired by the user and then the laser may be outputted.

According to an exemplary embodiment, the handpiece 330b may further include the SELIBS film 310.

The SELIBS film 310 may be detachably connected to the distal end of the detachable handpiece 330b for diagnosing a disease and measuring a skin age. For example, the SELIBS film 310 may be detachably connected with the ring 337. Referring back to FIG. 6, the SELIBS film is detachably connected to the ring.

Alternatively, the SELIBS film 310 may be placed on the body tissue without being connected to the detachable handpiece 330b.

In the above example described with reference to FIGS. 9A, 9B, and 10, the handpiece 330b may receive a collimated laser. Alternatively, the handpiece 330b may be configured to receive focused laser light rather than a collimated laser. For example, the handpiece 330b may be configured to include a collimator (not shown) (in this case, the collimator may be located in the first part). In this configuration, the handpiece 330b may receive a focused laser rather than a collimated laser from the laser irradiation device, and may convert the focused laser to a collimated laser and project the collimated laser onto the body tissue.

While exemplary embodiments have been particularly shown and described above, it will be understood by those of ordinary skill in the art that various changes in form and details may be made therein without departing from the spirit and scope of the present invention as defined by the following claims.

What is claimed is:

1. A system comprising:
a laser irradiation device configured to generate a laser and project the laser onto body tissue;
a probe configured to collect light which is generated when the laser is projected onto the body tissue; and
an analysis unit configured to diagnose a disease by analyzing a spectrum of the light collected by the probe,
wherein the analysis unit comprises:
a spectrometer configured to measure the spectrum of the light collected by the probe;
a first storage configured to store a disease diagnose reference spectrum data DB; and
a disease diagnosis unit configured to determine whether there is a disease in the body tissue based on the spectrum measured by the spectrometer with reference to the disease diagnosis reference spectrum data DB, and
wherein the probe is detachably connected to the laser irradiation device.

2. The system of claim 1, further comprising an SELIBS (Surface Enhanced Laser Induced Breakdown Spectroscopy) film, and
wherein the SELIBS film is connected to the laser irradiation device and increases an intensity of the generated light.

3. The system of claim 1, wherein the analysis unit is configured to measure a skin age by analyzing the spectrum of the light collected by the probe.

4. The system of claim 3, wherein the analysis unit comprises:
a second storage configured to store a skin age measurement reference spectrum data DB; and
a skin age measurement unit configured to measure a skin age of the body tissue based on the spectrum measured by the spectrometer with reference to the skin age measurement reference spectrum data DB.

5. The system of claim 1, wherein the analysis unit comprises a diagnosis server and a diagnosis terminal which are connected with each other via a network,
wherein the diagnosis server comprises the disease diagnosis unit and the second storage configured to store a disease diagnosis reference spectrum data DB,
wherein the diagnosis terminal comprises the spectrometer, and
wherein the diagnosis terminal is configured to transmit the spectrum measured by the spectrometer to the diagnosis server, and the diagnosis server is configured to determine whether there is a disease in the body tissue based on the spectrum measured by the spectrometer with reference to the disease diagnosis reference spectrum data DB, and transmit a result of the determining to the diagnosis terminal.

6. The system of claim 5, wherein the diagnosis server is configured to measure a skin age by analyzing the spectrum measured by the spectrometer, and
wherein the diagnosis server comprises:
a third storage configured to store a skin age measurement reference spectrum data DB; and
a skin age measurement unit configured to measure the skin age of the body tissue based on the spectrum measured by the spectrometer with reference to the skin age measurement reference spectrum data DB.

7. The system of claim 2, wherein an adhesive is formed on at least one surface of the SELIBS film to temporarily attach the SELIBS film to the body tissue.

8. The system of claim 2, wherein the laser irradiation device comprises a laser body and a handpiece, and
wherein the SELIBS film is connected to a distal end of the handpiece.

9. The system of claim 8, wherein a guide part is formed at the distal end of the handpiece for user convenience, and the SELIBS film is connected to the guide part.

10. The system of claim 2, wherein the SELIBS film comprises a substrate and a layer which is temporarily or fixedly formed on a surface of the substrate to increase the intensity of the generated light.

11. The system of claim 1, wherein the laser irradiation device is a low level pulsed laser-based aesthetic or medical laser irradiation device using a collimated laser.

12. The system of claim 2, wherein the laser irradiation device is a low level pulsed laser-based aesthetic or medical laser irradiation device using a collimated laser.

13. A handpiece detachably connected to a laser irradiation device, the handpiece comprising:
a body part which is formed in a cylindrical shape to have a path formed therein to allow light to move therein, and which comprises an entrance part for receiving light generated when light is projected by the laser irradiation device onto body tissue, and a connection part which is attachable to or detachable from the laser irradiation device; and
an optical unit which is configured to provide the generated light entering through the entrance part to a spectrometer.

14. The handpiece of claim 13, wherein the optical unit comprises a first optical module, a second optical module, and a third optical module, and
wherein the first optical module is configured to change a direction of at least part of the generated light entering through the entrance part, the second optical module is configured to re-change the direction of the generated light of which the direction has been changed by the first optical module, and the third optical module is configured to provide the generated light of which the direction has been re-changed by the second optical module to an optical fiber connected with the spectrometer.

15. The handpiece of claim 14, wherein the body part comprises a first part having a space for allowing light to move therein, and a second part having a space for allowing light to move therein, and
wherein the first optical module is located in the first part and the second optical module an the third optical module are located in the second part.

16. The handpiece of claim 15, wherein the first part is located in a center of the body part and the second part is formed to enclose the first part.

17. The handpiece of claim 13, further comprising an SELIBS film,
wherein the SELIBS film is disposed in contact with the body tissue,
wherein the light generated when the light is projected by the laser irradiation device onto the body tissue enters the entrance part via the SELIBS film, and
wherein the SELIBS film is configured to increase the intensity of the generated light.

18. The handpiece of claim 17, further comprising a guide part connected with the body part, for allowing the light projected by the laser irradiation device to be aligned with the body tissue, and
wherein the SELIBS film is detachably connected to the guide part.

19. The handpiece of claim 17, wherein the SELIBS film comprises a substrate and a layer which is temporarily or fixedly formed on a surface of the substrate, for increasing the intensity of the generated light.
